(19) United States
(12) Patent Application Publication
(10) Pub. No.: US 2026/0217633 A1
(43) Pub. Date: Jul. 30, 2026

(54) SYSTEMS AND METHODS FOR E-METHANOL PRODUCTION

(71) Applicant: SAUDI ARABIAN OIL COMPANY, Dhahran (SA)

(72) Inventors: Ghassan S. ALSHEHRY, Dhahran (SA); Hassan Sakar ALQAHTANI, Dhahran (SA); Khalid Mohammed ALRUWAILI, Dhahran (SA); Saad Menahi AL MUTAIRI, Dhahran (SA)

(73) Assignee: SAUDI ARABIAN OIL COMPANY, Dhahran (SA)

(21) Appl. No.: 19/038,851

(22) Filed: Jan. 28, 2025

(51) Int. Cl. C07C 29/151; C01B 3/24; C25B 1/04; C25B 15/08

(52) U.S. Cl. CPC C07C 29/1518; C01B 3/24; C25B 1/04; C25B 15/081; C01B 2203/049; C01B 2203/061; C01B 2203/1241

(57) ABSTRACT

Systems and methods for E-methanol production may comprise capturing carbon dioxide from an exhaust gas, the exhaust gas being a byproduct of a gas processing plant; hydrogenating the carbon dioxide with a hydrogen gas to produce a syngas; synthesizing a stream comprising methanol and water from the syngas; and separating at least a portion of the water from the stream to produce an E-methanol.

### FIELD OF THE DISCLOSURE

The present disclosure relates generally to E-methanol production and, more particularly, to E-methanol production from carbon dioxide emissions and wastewater from gas processing plants.

### BACKGROUND OF THE DISCLOSURE

Global concerns over climate change have intensified efforts to reduce anthropogenic carbon dioxide emissions. The Paris Agreement, adopted in 2015, set a framework for limiting global temperature rise to well below 2° C., with the goal of reaching net-zero emissions in the second half of the current century. The oil and gas sector, a major contributor to greenhouse gas emissions, is under increasing pressure to develop technologies that capture and utilize carbon dioxide rather than vent it into the atmosphere.

The decarbonization of gas processing facilities has become a critical area of focus. Current carbon capture technologies, including direct air capture (DAC) and carbon capture, utilization, and storage (CCUS), are effective but often capital-intensive, particularly when applied to highly acidic gas streams. Furthermore, the separation of carbon dioxide during gas sweetening, a common process in gas plants, typically results in the venting of carbon dioxide into the atmosphere, contributing to overall emissions. In response to these challenges, various efforts have been made to develop cost-effective and scalable methods for capturing and repurposing carbon dioxide emissions from gas plants.

Hydrogen plays a crucial role in the conversion of carbon dioxide into value-added products, such as synthetic fuels and chemicals. Hydrogen production methods, including electrolysis and methane pyrolysis, offer pathways to generating “green” and “turquoise” hydrogen with minimal environmental impact. Green hydrogen, produced through the electrolysis of water, is often favored for its sustainability but remains expensive due to the energy-intensive nature of the process. Turquoise hydrogen, produced through methane pyrolysis, has emerged as a lower-cost alternative that generates solid carbon as a byproduct, avoiding carbon dioxide emissions.

Methanol, a liquid chemical with numerous industrial applications, is gaining attention as a potential pathway for carbon dioxide utilization. Methanol may be synthesized from syngas, a mixture of carbon monoxide, hydrogen, and carbon dioxide, through conventional catalytic process. It serves as a versatile feedstock for the chemical industry and may be used as a fuel in various energy applications. Moreover, methanol is easier and safer to store and transport compared to gaseous hydrogen or carbon dioxide, making it a suitable candidate for large-scale carbon utilization.

Gas processing plants, in addition to emitting carbon dioxide, produce vast quantities of wastewater as part of their operations. This wastewater, which arises from various stages of gas production and processing is typically treated as a waste product, is often disposed of through evaporation ponds or other means. The potential to repurpose this wastewater for hydrogen production, particularly through hydrolysis, represents an opportunity to enhance the sustainability of gas plant operations.

There remains a need for processes that may effectively integrate carbon dioxide capture, hydrogen production, and methanol synthesis using existing resources at gas processing facilities. Such processes may not only mitigate emissions but also create value from waste streams, contributing to the overall decarbonization of the oil and gas industry.

### SUMMARY OF THE DISCLOSURE

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, methods for E-methanol production may include capturing carbon dioxide from an exhaust gas, the exhaust gas being a byproduct of a gas processing plant; hydrogenating the carbon dioxide with a hydrogen gas to produce a syngas; synthesizing a stream comprising methanol and water from the syngas; and separating at least a portion of the water from the stream to produce an E-methanol.

In another embodiment, systems for E-methanol production may include an amine adsorption unit configured to capture carbon dioxide from an exhaust gas, the exhaust gas being a byproduct of a gas processing plant; a hydrogen production unit comprising a wastewater electrolysis unit and a methane pyrolysis unit; a syngas reactor configured to hydrogenate the carbon dioxide captured from the exhaust gas; a methanol reactor configured to synthesize a stream comprising methanol and water from the syngas; and a distillation column configured to separate at least a portion of the water from the stream.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

Not applicable.

### DETAILED DESCRIPTION

Embodiments in accordance with the present disclosure generally relate to E-methanol production and, more particularly, to E-methanol production from carbon dioxide emissions and wastewater from gas processing plants. The present disclosure provides an approach for converting carbon dioxide emissions and wastewater generated by gas processing plants into E-methanol, a valuable chemical and fuel, thereby addressing critical environmental challenges. Gas processing plants are significant contributors to global carbon dioxide emissions and wastewater production. The present disclosure leverages these waste streams to produce methanol onsite, offering a potentially economically and environmentally favorable solution to mitigate the carbon footprint of gas plants.

The systems and methods described herein involve capturing carbon dioxide from gas plant emissions using amine adsorption, a well-established technique that may efficiently produce high-purity carbon dioxide streams. This captured carbon dioxide may undergo hydrogenation to form syngas, a mixture of carbon monoxide, hydrogen, and carbon dioxide, which may be subsequently converted into methanol via conventional reaction processes. The final separation of methanol from water may be achieved through a single distillation column, a commercially proven technology widely used in industrial applications. This may potentially ensure the reliability and scalability of the process for large-scale deployment.

A distinguishing factor of the systems and methods herein pertain to the hydrogen sourcing. Hydrogen, essential for the hydrogenation of carbon dioxide, may be generated from two primary sources: purified wastewater and excess natural gas. The wastewater, which may be a byproduct of gas plant operations, may undergo purification and electrolysis to produce green hydrogen. Electrolysis may involve splitting water into hydrogen and oxygen using electrical energy, thereby considered a sustainable hydrogen production method. Additionally, excess natural gas from the gas plant may be utilized to produce turquoise hydrogen via methane pyrolysis, a process that relies on the decomposition of methane into hydrogen and solid carbon. Methane pyrolysis may be advantageous due to its low energy requirements and the absence of carbon dioxide emissions, further enhancing the sustainability of the methods described in the present disclosure.

The systems and methods of the present disclosure may potentially offer several advantages over existing carbon dioxide-to-methanol conversion methods. For example, the systems and methods described herein utilize waste streams (e.g., carbon dioxide, wastewater, and excess natural gas) that are already present at the gas plant, reducing the need for external resources and infrastructure. This may minimize both operational costs and the environmental footprint associated with transporting carbon dioxide or hydrogen. In contrast, many conventional methods for E-methanol production rely on more costly and less efficient carbon dioxide capture techniques, including direct air capture (DAC), which may require additional infrastructure investment. Moreover, the use of onsite resources may eliminate the need for long-distance transportation of gaseous carbon dioxide, which may otherwise necessitate significant energy input and pipeline infrastructure.

Another potential advantage of the systems and methods described herein may be the reduced cost of hydrogen production. Hydrogen is historically one of the most expensive components in carbon dioxide hydrogenation processes. The present disclosure may address the high cost of hydrogen by utilizing two lower-cost sources: green hydrogen from wastewater electrolysis and turquoise hydrogen from methane pyrolysis. While green hydrogen is generally expensive due to the high energy demands of electrolysis, this process may offset some of these costs by utilizing wastewater that would otherwise be discarded. Turquoise hydrogen, produced via methane pyrolysis, may further reduce costs, as it consumes less energy than electrolysis and generates solid carbon as a byproduct, which may be sold or used in other industrial applications. This dual hydrogen-sourcing strategy may provide a significant cost advantage over processes that rely solely on blue or green hydrogen, both of which are more resource-intensive.

From an environmental perspective, the systems and methods of the present disclosure may potentially offer significant benefits. By capturing carbon dioxide emissions and converting them to methanol, the process may directly reduce the carbon emissions associated with gas processing. Methanol, a liquid fuel that may be easily stored and transported, is a valuable commodity that may be used in various industrial and energy applications. Furthermore, the use of turquoise hydrogen, which generates solid carbon as a byproduct rather than carbon dioxide, potentially adds an additional layer of sustainability. The solid carbon may be further monetized or utilized in other industries, creating a potential revenue stream and reducing waste.

The scalability of the system and methods described herein may potentially provide another advantage. Because the systems and methods of the present disclosure rely on conventional technologies such as amine adsorption for carbon dioxide capture, electrolysis for green hydrogen production, and methane pyrolysis for turquoise hydrogen, these methods may potentially be implemented at existing gas plants with minimal modifications. Many gas plants already employ carbon dioxide capture technologies as part of the gas sweetening process, meaning that the infrastructure for carbon dioxide is already in place. The integration of hydrogen production from wastewater and excess natural gas further enhances the feasibility of scaling this process to meet the needs of large gas processing facilities. The ability to scale the process without significant new infrastructure investments may make it a viable solution for decarbonizing the oil and gas industry.

Therefore, methods for E-methanol production may comprise capturing carbon dioxide from an exhaust gas, the exhaust gas being a byproduct of a gas processing plant; hydrogenating the carbon dioxide with a hydrogen gas to produce a syngas; synthesizing a stream comprising methanol and water from the syngas; and separating at least a portion of the water from the stream to produce an E-methanol.

Furthermore, systems for E-methanol production may comprise an amine adsorption unit configured to capture carbon dioxide from an exhaust gas, the exhaust gas being a byproduct of a gas processing plant; a hydrogen production unit comprising a wastewater electrolysis unit and a methane pyrolysis unit; a syngas reactor configured to hydrogenate the carbon dioxide captured from the exhaust gas; a methanol reactor configured to synthesize a stream comprising methanol and water from the syngas; and a distillation column configured to separate at least a portion of the water from the stream.

The systems and methods of the present disclosure involve the capture of carbon dioxide from an exhaust gas of a gas processing plant comprising, for example, a gas sweetening process. The exhaust gas may, for example, have a carbon dioxide concentration of about 1 wt % to about 20 wt % (or about 1 wt % to about 15 wt %, or about 1 wt % to about 10 wt %, or about 1 wt % to about 8 wt %, or about 5 wt % to about 20 wt %, or about 5 wt % to about 15 wt %, or about 5 wt % to about 10 wt %, or about 5 wt % to about 8 wt %, or about 8 wt % to about 20 wt %, or about 8 wt % to about 15 wt %, or about 8 wt % to about 10 wt %). Capturing the carbon dioxide from the exhaust gas may comprise amine adsorption using an amine adsorption unit, which may be units conventionally used in gas sweetening processes. In the amine adsorption, amine-based solvents may be used to adsorb carbon dioxide from the exhaust gas, thereby producing a carbon dioxide-laden solvent. The carbon dioxide-laden solvent may then be subjected to heating to release a high-purity carbon dioxide, which may be collected for further processing.

The carbon dioxide captured from the exhaust gas may be hydrogenated with a hydrogen gas to produce a syngas. Optionally, the hydrogen gas may comprise a green hydrogen, a turquoise hydrogen, or a combination thereof. In the instance that green hydrogen is utilized, the green hydrogen may be produced from a wastewater generated in the gas processing plant. The wastewater may be the result of processes such as cooling, heating, and/or other utility functions and may be collected and/or purified to remove contaminants. Once purified, the wastewater may be introduced into an electrolysis unit. The electrolysis of the wastewater may split at least a portion of the wastewater into hydrogen (H₂) and oxygen (O₂) using electrical energy.

In the instance that turquoise hydrogen is utilized, the turquoise hydrogen may be produced, for example, through methane pyrolysis, which may potentially utilize an excess natural gas produced by the gas processing plant. During methane pyrolysis, methane may be thermally decomposed in the absence of oxygen to form hydrogen, solid carbon, or a combination thereof. The pyrolysis of methane may consume about 10 kWh to about 20 kWh of energy per kilogram of hydrogen produced (or about 10 kWh/kg H₂ to about 15 kWh/kg H₂, or about 15 kWh/kg H₂ to about 20 kWh/kg H₂). The solid carbon produced during the methane pyrolysis may be further processed for use in various industrial applications.

In any embodiment, the captured carbon dioxide may be hydrogenated to produce a syngas, which may include, but is not limited to, carbon monoxide, hydrogen, carbon dioxide, the like, and any combination thereof. The hydrogenation reaction may occur in a reactor in which the CO₂ interacts with the hydrogen gas at an elevated temperature and pressure in the presence of a suitable catalyst. Suitable catalysts may include, but are not limited to, copper-based catalysts, zinc oxide catalysts, the like, and any combination thereof.

After a syngas is produced, the syngas may be used to synthesize methanol and water in a methanol reactor. The methanol reaction may take place under elevated temperatures and pressures and optionally in the presence of a suitable catalyst (e.g., a copper-based catalyst and/or a zinc oxide catalyst). The interaction of the carbon monoxide and carbon dioxide with hydrogen may produce methanol and water.

Following the synthesis of the stream comprising methanol and water, distillation may be utilized to separate at least a portion of the water from the stream. For example, a distillation column may be employed to produce an E-methanol. The distillation process may advantageously rely on the differing boiling points of methanol and water by heating the components in the column, resulting in the vaporization of the E-methanol. The E-methanol may be captured and stored for further use as a chemical feedstock.

The present disclosure is further directed to the following non-limiting clauses:
Clause 1. A method comprising:
   capturing carbon dioxide from an exhaust gas, the exhaust gas being a byproduct of a gas processing plant;
   hydrogenating the carbon dioxide with a hydrogen gas to produce a syngas;
   synthesizing a stream comprising methanol and water from the syngas; and
   separating at least a portion of the water from the stream to produce an E-methanol.
Clause 2. The method of clause 1, wherein the carbon dioxide is captured from the exhaust gas using amine adsorption.
Clause 3. The method of clause 1 or clause 2, wherein the hydrogen gas comprises green hydrogen, turquoise hydrogen, or a combination thereof.
Clause 4. The method of clause 3, wherein the hydrogen gas comprises green hydrogen, and the method further comprises using electrolysis to produce the green hydrogen from a wastewater.
Clause 5. The method of clause 3, wherein the hydrogen gas comprises turquoise hydrogen, and the method further comprises pyrolyzing methane to produce the turquoise hydrogen.
Clause 6. The method of clause 5, wherein the pyrolyzing also produces a solid carbon.
Clause 7. The method of clause 6, further comprising processing the solid carbon.
Clause 8. The method of clause 5, wherein the pyrolyzing consumes about 10 kWh to about 20 kWh of energy per kilogram of hydrogen produced.
Clause 9. The method of any one of clauses 1-8, wherein hydrogenating the carbon dioxide comprises interacting the carbon dioxide with the hydrogen gas in the presence of a catalyst.
Clause 10. The method of clause 9, wherein the catalyst comprises a copper-based catalyst, a zinc oxide catalyst, or a combination thereof.
Clause 11. The method of any one of clauses 1-10, wherein separating at least a portion of the water from the stream comprises distillation.
Clause 12. The method of any one of clauses 1-11, wherein the exhaust gas has a concentration of carbon dioxide of about 1 wt % to about 20 wt %.
Clause 13. The method of any one of clauses 1-12, wherein the gas processing plant comprises a gas sweetening process.
Clause 14. The method of any one of clauses 1-13, further comprising utilizing the E-methanol as a chemical feedstock.
Clause 15. A system comprising:
   an amine adsorption unit configured to capture carbon dioxide from an exhaust gas, the exhaust gas being a byproduct of a gas processing plant;
   a hydrogen production unit comprising a wastewater electrolysis unit and a methane pyrolysis unit;
   a syngas reactor configured to hydrogenate the carbon dioxide captured from the exhaust gas;
   a methanol reactor configured to synthesize a stream comprising methanol and water from the syngas; and
   a distillation column configured to separate at least a portion of the water from the stream.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains,” “containing,” “includes,” “including,” “comprises,” and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

The invention claimed is:

1. A method comprising:
capturing carbon dioxide from an exhaust gas, the exhaust gas being a byproduct of a gas processing plant;
hydrogenating the carbon dioxide with a hydrogen gas to produce a syngas;
synthesizing a stream comprising methanol and water from the syngas; and
separating at least a portion of the water from the stream to produce an E-methanol.

2. The method of claim 1, wherein the carbon dioxide is captured from the exhaust gas using amine adsorption.

3. The method of claim 1, wherein the hydrogen gas comprises green hydrogen, turquoise hydrogen, or a combination thereof.

4. The method of claim 3, wherein the hydrogen gas comprises green hydrogen, and the method further comprises using electrolysis to produce the green hydrogen from a wastewater.

5. The method of claim 3, wherein the hydrogen gas comprises turquoise hydrogen, and the method further comprises pyrolyzing methane to produce the turquoise hydrogen.

6. The method of claim 5, wherein the pyrolyzing also produces a solid carbon.

7. The method of claim 6, further comprising processing the solid carbon.

8. The method of claim 5, wherein the pyrolyzing consumes about 10 kWh to about 20 kWh of energy per kilogram of hydrogen produced.

9. The method of claim 1, wherein hydrogenating the carbon dioxide comprises interacting the carbon dioxide with the hydrogen gas in the presence of a catalyst.

10. The method of claim 9, wherein the catalyst comprises a copper-based catalyst, a zinc oxide catalyst, or a combination thereof.

11. The method of claim 1, wherein separating at least a portion of the water from the stream comprises distillation.

12. The method of claim 1, wherein the exhaust gas has a concentration of carbon dioxide of about 1 wt % to about 20 wt %.

13. The method of claim 1, wherein the gas processing plant comprises a gas sweetening process.

14. The method of claim 1, further comprising utilizing the E-methanol as a chemical feedstock.

15. A system comprising:
an amine adsorption unit configured to capture carbon dioxide from an exhaust gas, the exhaust gas being a byproduct of a gas processing plant;
a hydrogen production unit comprising a wastewater electrolysis unit and a methane pyrolysis unit;
a syngas reactor configured to hydrogenate the carbon dioxide captured from the exhaust gas;
a methanol reactor configured to synthesize a stream comprising methanol and water from the syngas; and
a distillation column configured to separate at least a portion of the water from the stream.
